(19) United States
(12) Patent Application Publication
(10) Pub. No.: US 2010/0089409 A1
(43) Pub. Date: Apr. 15, 2010

(54) Postoperative Adhesion Prophylactic

(76) Inventor: Rudolf Bertagnoli, Straubing (DE)

Correspondence Address: ROBERT W. BECKER & ASSOCIATES, 707 HIGHWAY 333, SUITE B, TIJERAS, NM, 87059-7507, US

(21) Appl. No.: 12/574,155

(22) Filed: Oct. 6, 2009

(30) Foreign Application Priority Data
Oct. 6, 2008 (DE) 102008050122.0

(51) Int. Cl. A61B 19/08

(52) U.S. Cl. 128/850

(57) ABSTRACT

A method for postoperative use of an adhesion prophylactic. After an operation that might have to be repeated, a non-reabsorbable membrane, made of material that is compatible with a body, is placed between an operation site and adjacent tissue. The membrane is configured such that during a repair operation the membrane can be used to facilitate exposure of the operation site.

The present invention relates to a method for the postoperative use of an adhesion prophylactic, according to which subsequent to a surgical operation, an insert is placed between the location of operation and vessels and/or organs; the invention also relates to an operation aid for carrying out the method.

Surgical operations on the human body generally lead to scar tissue during the healing process, which can connect adjacent vessels, organs and/or tissue with the scarring site, which can not only cause the patient pain and discomfort, but can also lead to complications during a further operation or repair.

Pursuant to DE 196 00 095, after the surgical operation a foil that is re-absorbable by the body is placed between the operation site and adjacent tissue, etc. and prevents adhesion of tissue with the scar.

With implantations or other operations that require repair, such an insert could be an obstruction or at the least is not helpful. For example, during the implantation of vertebral body or intervertebral disc prosthesis in the lumbar region of the abdominal cavity, the organs and vessels that lie in front of the spinal column must be shifted and held to the side during the implantation. Although an insert pursuant to the state of the art prevents adhesion of the organs with the spinal column after following the operation, such an insert provides no facilitation when a repair operation is required.

It is an object of the present invention to improve the adhesion prophylactic with respect to repair operations.

This object is inventively realized by the features of claims 1 and 6.

By inserting a non re-absorbable membrane made of material that is compatible with the body between the operation site and adjacent tissue, organs, vessels, not only is the patient spared pain and discomfort, at the same time an operation aid for a further surgical operation is provided. The membrane that is inserted in the initial operation is used in a follow-on operation as a support for the shifting of tissue, such as organs and vessels, in order to expose the operation site for the surgeon.

The membrane is made of known materials that are compatible with the body, such as carbon fibers, polymeric materials that are compatible with the body, etc., and can have a number of different designs. Depending upon the information that is available, membranes can be used that are smooth on both sides or are rough on both sides, or that have one smooth and one rough surface. Similarly, double-layered or multi-layered membranes can be used.

The rough side permits a growing together with tissue, whereas the smooth side is intended to prevent this type of adhesion.

For the reliable fixation of the membrane, staples can be used that either permanently remain in the body or are re-absorbable. It is possible to permanently anchor a base or support to bone material on which the membrane is detachably connected, for example, via screws, hooks or similar means. Alternatively, the membrane is held in position by being sewn to tissue.

It is expedient to use re-absorbable staples, which as opposed to sewing accelerates the operation process and which disappear with time and do not cause the patient any pain. In order in this case to prevent a subsequent slipping of the membrane, the membrane preferably has a rough configuration on at least one side to enable growing together with tissue, thereby stabilizing the position of the membrane.

Pursuant to a further embodiment of the invention, the smooth side of the membrane is directed toward the operation site, for example an implantation site, in order to prevent adhesion of the membrane with the operation site or prosthesis, and hence to avoid an encroachment of the scar site or operation site. The membrane grows together with the organs and possibly vessels by means of the rough side. This configuration has the further advantage that in the event of a repair or necessary further operation, the total package of organs, vessels and membrane are easier to handle and/or to shift in order to expose the operation site.

Pursuant to a further embodiment of the invention, the membrane is produced from a combination of materials such that the membrane has different properties or characteristics on its two sides. These different characteristics can also be realized by two appropriately differently designed membranes that are laminarly connected to one another. With such a configuration of the membrane, it is possible to achieve an optimum adaptation on the one hand to the scar and the other hand to the tissue, organs and/or vessels.

With an appropriate indication or information, the membrane can readily be inserted in the reverse manner, namely with the smooth side facing the organs and the rough side facing the operation site.

To produce different growing-together properties, the membrane can also be appropriately coated on one side.

Pursuant to a further embodiment of the invention, the membrane is comprised of two superimposed membranes or foils having the same or different growing-on or together characteristics. This enables an optimum adaptation to the respective actual condition. The two membranes can be laminarly connected to one another, or can also be comprised of two loose membranes that are either not connected to one another or are connected at only one edge. Inflatable membranes can also be used that have a closeable connection via which air is withdrawn after the operation and air is blown in for a further operation. During the inflation, the tissue is pressed away and the access to the operation site is effected via the appropriately embodied connection by means of instruments that are used with minimally invasive operations. By means of an external hollow needle, air can be constantly blown in in order to maintain the expanded state of the membrane.

A further embodiment of the invention provides for a multi-layered, in particular three-layered, membrane. The intermediate layer is embodied in such a way that it prevents a collection of fluid, a serum formation, and hence reduces the risk of infection. In the event of a repair operation, the intermediate layer at the same time serves as a separation means for the two outer membrane layers that are adjacent to the tissue. In this connection, the multi-layered membrane is configured such that when the membrane is inserted in the body of the patient, the intermediate layer or layers can be separated from the membrane layers, so that the two outer membrane layers that are thereby separated can be readily removed and/or separated from one another. In this form, they serve for the clean separation from tissue and operation site, which provides the surgeon not only rapid access to the operation site, but also a more straightforward operation as such. In this connection, it is immaterial whether the outer sides of the two membrane layers have grown together with the adjoining tissue or not. This design thus ensures a clean and reliable separation of the membrane layers in order to rapidly expose the operation site for the repair.

The intermediate layer or layers are detachably connected with the outer membrane layers in any technically feasible manner. Detachable adhesive connections, hook and loop closures can be used.

A ring configuration is also suitable as an intermediate layer, and separates only the edge regions of the two outer membrane layers from one another.

One advantageous embodiment comprises a three-layered membrane, the outer sides of which permit a growing onto tissue and the intermediate layer of which is removable and can again be inserted. This offers the surgeon a cleanly defined operation site after the separation and/or turning up of the two membrane layers in that the membrane covers the surrounding area of the operation site, while for the operation site an opening is provided in the membrane layer that faces the operation site and that is covered with a removable window cover.

The inventive membrane, for an adhesion prophylactic, can be used anywhere where a growing together between tissue and operation site is to be prevented, and where a further operation is possible, for example in connection with intestinal diseases, cyst formation, implantations, etc.

Pursuant to a further embodiment, the membrane is part of a tension cord band, or is connected therewith. This is expedient with spinal or intervertebral disc operations, where for example a tension cord band must be introduced. In this connection, the membrane, which is connected along the tension cord band, can be produced from any material that is compatible with the body. The tension cord band can also be formed directly by the membrane.

The invention will be described in detail with the aid of examples schematically illustrated in the drawings, in which:

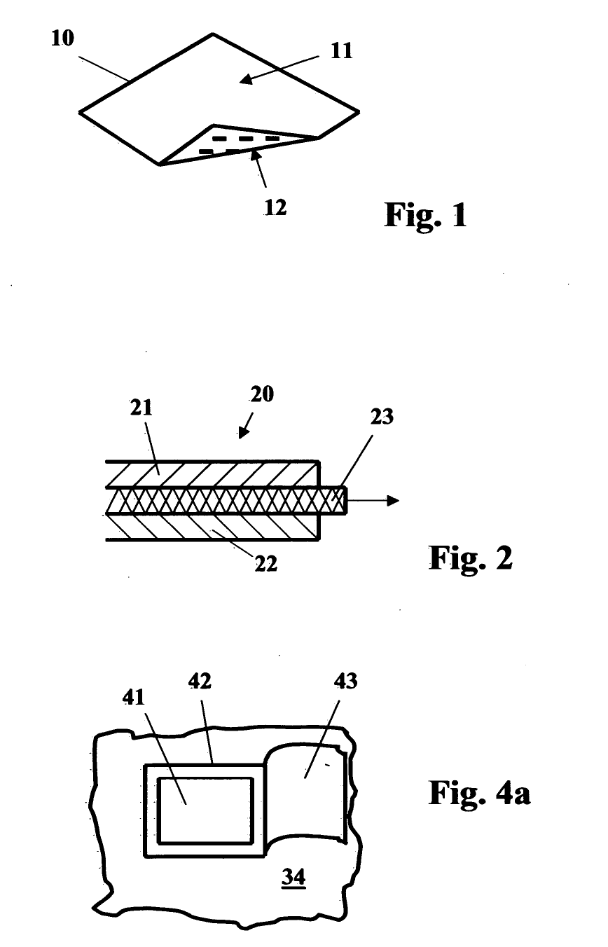

FIGS. 1 and 2 each show one embodiment,

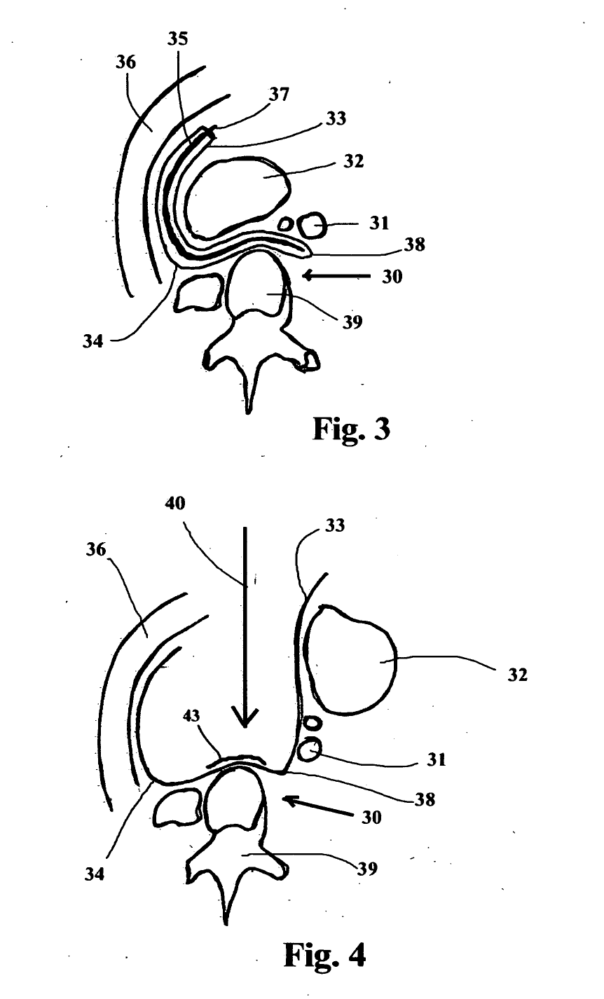

FIGS. 3 and 4 represent the use with initial and subsequent operations respectively,

FIG. 4a shows a portion from FIG. 4, and

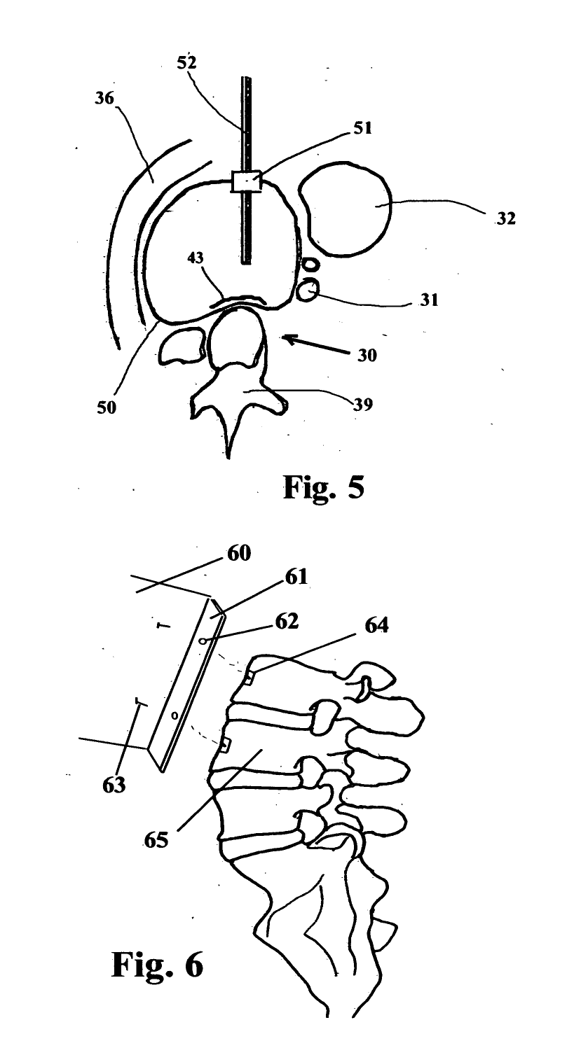

FIGS. 5 and 6 each show a further embodiment.

Pursuant to FIG. 1, one inventive adhesion prophylactic is comprised of a membrane 10 that is made of a material that is compatible with the body; in the illustrated embodiment, the membrane has a smooth side 11 and a rough or textured side 12. The purpose of this embodiment is that the membrane can grow together with the tissue on only one side. The unequal characteristics can be achieved by appropriate design of a single-layer or dual-layer membrane, or by coating one or both sides of the membrane. Depending upon the application, membranes can also be used that have the same properties or characteristics on both sides, i.e. both sides are smooth or rough.

Different characteristics can also be produced by means of dual-layered membranes where the different characteristics are associated with the respective membrane layer. A connection of the two layers is effected by a stick-type or hook and loop connection, a detachable adhesive layer, or the like.

FIG. 2 shows an example having a three-layered membrane 20, whereby an intermediate layer 23 on the one hand separates the two actual membrane layers 21, 22 and on the other hand detachably connects the actual layers in such a way that when in a position where it is placed in the body of the patient, the intermediate layer 23 is removable in the direction of the arrow and thereby frees the two outer membrane layers 21, 22 for being opened or turned up. The separation is generally effected by pulling off the upper layer 21, where-upon the intermediate layer 23 is then removed. The intermediate layer is, for example, a detachable adhesive layer, a layer that is connectable with the outer layers via a hook and loop connection, or the like.

The two membrane layers 21, 22 can advantageously be connected to one another at an edge, and can open up like a book after the removal of the intermediate layer 23, as shown in FIG. 4 with the membrane layers 33, 34. In this way, the membrane layers expose the site 30 of the operation, whereby at the same time they cover the tissue sites 31, 32, 36, 39 (FIG. 4) near the location of the operation. For the operation site 30 as such, an opening 41 is provided in the membrane layer 34 that is on the operation side (FIG. 4a); The opening can be releasably closed off by a window cover 43. For this purpose, a detachable adhesive layer or hook and loop strips 42 can be provided about the opening 41.

The inventive method will be described with the aid of FIGS. 2, 3 and 4a in conjunction with an example of an implantation of a vertebral body replacement or an intervertebral disc prosthesis, which is carried out at the front side. FIGS. 3 and 4 show a cross-section through the lumbar region of a patient, whereby FIG. 3 represents that state after the first operation and FIG. 4 represents the preparation for a repair operation.

With such an operation, vessels 31 and/or organs 32 that lie in front of the spinal column 39 or the operation site 30 must be shifted to the side and held.

In the first operation, after successful implantation of the prosthesis, a membrane 33-35 is placed over the treated spinal location 30 and is appropriately fixated, for example by means of staples, and thereafter the organs 32 and vessels are again placed back. This state is illustrated in FIG. 3, where the membrane 33-35 comes to rest between the operation site 30 and the vessels/organs 31, 32, and between the latter and the peritoneum 36. This prevents the organs 32 and vessels 31 from growing together with the scar tissue at the operation site 30 and causing pain or discomfort to the patient. In particular in case of a renewed operation the membrane 33-35 serves to considerable simplify at least the preparation for the actual operation. In the example of FIGS. 3 and 4, a multi-layered membrane, as was described in conjunction with FIG. 2, is being used. The intermediate layer 35 is detachably connected with the two membrane layers 33, 34 by means of detachable or releasable adhesive, a hook and loop closure, or the like. The intermediate layer 35 is embodied in such a way that it prevents the formation of serum between the two membrane layers in order to reduce the risk of infection.

If a repair operation is to be undertaken, the intermediate layer 35 is withdrawn or separated from connection to the two respective membrane layers 33, 34 by means of its projecting end 37. After removal or separation of the intermediate layer 35, the two membrane layers 33, 34 can effortlessly be separated from one another and be opened or turned up, whereby via the membrane layer 33 the vessels 31 and organs 32 can be shifted to the side as a unit, so that the surgeon obtains a free access 40 to the operation site 30.

The two outer membrane layers 33, 34 are connected together along an edge 38, which forms a fold edge.

For the access to the operation site 30, an opening 41, FIG. 4a, is provided in the lower membrane layer 34; this opening is exposed by removing the window cover 43. This concludes the preparations for the actual operation, e.g. the replacement of an intervertebral disc prosthesis.

After the repair operation, for example the replacement of the intervertebral disc prosthesis, the window cover 43, or a new window cover, is placed over the adhesive or stick-together site 42 and is connected with the membrane layer 34, where-upon the intermediate layer 35 or a new intermediate layer is again introduced and is detachably connected with the membrane layers 33, 34.

FIG. 5 shows an inflatable membrane 50, the interior of which is evacuated or inflated by means of a closeable connection 51. After the initial operation, the evacuated membrane bladder 50 is inserted as a flat, double-walled membrane, as illustrated in FIG. 3. Prior to a further operation, the membrane bladder 50 is inflated via the connection 51. The inner pressure shifts the tissues 31, 32 to the side, so that, as in the example of FIG. 4, access is provided to the operation site 30. By means of the tubes and instruments 52 guided through the connection 51, the window cover 43 is folded or flipped up and the operation can be undertaken in the region 30. The air pressure within the membrane 50 is maintained during the operation. Two connections, one for the air pressure and one for the instruments, can also be provided. After the conclusion of the operation, the window cover 43 is folded down and the interior is again evacuated.

The fixation of a membrane is effected, to the extent necessary, depending upon use, by sewing, with re-absorbable staples, or by means of securement plates 64 that are fixed to bone 65 (FIG. 6), or the like.

FIG. 6 shows an example where the membrane 60 is connected with a tension cord band 61 for the spine, or forms a unit. The vertebral bodies, between which for example a non-illustrated artificial disc was placed, are held together be means of the tension cord band 61. The tension cord band 61 is part of the membrane 60 and also fulfills the function of a membrane. For the detachable fixation of the membrane 60, apertures 62 are provided in the tension cord band 61 via which is effected a securement by means of screws 63 and the nuts 64 that are fixedly and permanently anchored in the bone material.

The formation of serum between two membrane layers is also prevented in that the two membrane layers are detachably directly adhesively connected to one another without an intermediate layer. The use of a single-layer membrane is effected in a similar manner, as previously described. In the example of FIG. 4, the single-layered membrane, for example together with the organs/vessels 31, 32, is held up similar to the membrane 33, thereby making the operation site 30 directly accessible, since the second membrane layer 34 is missing. In this case, the membrane edge 38 can be detachably connected with the vertebral bodies 39, as shown in FIG. 6

1-12. (canceled)

13. A method for postoperative use of an adhesion prophylactic, including the step of:
after an operation that might have to be repeated, placing a non-reabsorbable membrane between an operation site and adjacent tissue, wherein said membrane is configured such that during a repair operation said membrane is adapted to be used to facilitate exposure of the operation site.

14. A method according to claim 13, wherein with an implantation of a prosthesis or implant, said placing step comprises placing said membrane between adjacent tissue and the operation site or prosthesis such that during a repair operation, said membrane is adapted to be shifted together with the tissue for exposure of the operation site.

15. A method according to claim 13, wherein with an implantation of a vertebral body prosthesis or intervertebral disc prosthesis, said placing step comprises placing said membrane between the operation site or the prosthesis, and organs and vessels disposed in front of the operation site or prosthesis, such that during a repair operation the organs and vessels can be shifted together with said membrane for exposure of the operation site.

16. A method according to claim 13, wherein said membrane is a multi-layer membrane that includes at least one intermediate layer and outer membrane layers, and wherein during a repair operation said at least one intermediate layer is adapted to be removed or pulled out to separate said outer membrane layers.

17. A method according to claim 13, which includes the further step of fixating said membrane with reabsorbable staples.

18. An operation aid, comprising:
a membrane made of material that is compatible with a body, wherein said membrane is configured as an adhesion prophylactic for placement between a surgical operation site and adjacent tissue such that during a repair operation said membrane is adapted to be used to aid in exposure of the operation site.

19. An operation aid according to claim 18, wherein said membrane is comprised of two superimposed membranes.

20. An operation aid according to claim 18, wherein said two superimposed membranes have different characteristics.

21. An operation aid according to claim 19, wherein said two superimposed membranes are connected to one another along one edge.

22. An operation aid according to claim 19, wherein an intermediate layer is provided between said two superimposed membranes, and wherein during a repair operation said intermediate layer ensures a reliable separation of said two superimposed membranes.

23. An operation aid according to claim 18, wherein said membrane is an inflatable membrane.

24. An operation aid according to claim 18, wherein said membrane is part of a tension cord band or is connected to a tension cord band.
